(19) United States
(12) Patent Application Publication
(10) Pub. No.: US 2013/0236538 A1
(43) Pub. Date: Sep. 12, 2013

(54) PHARMACEUTICAL COMPOSITIONS OF IBUPROFEN AND FAMOTIDINE

(71) Applicant: HORIZONPHARMA USA, INC., Deerfield, IL (US)

(72) Inventors: George F. Tidmarsh, Portola Valley, CA (US); Iain W. Duncan, Seattle, WA (US)

(21) Appl. No.: 13/849,213

(22) Filed: Mar. 22, 2013

Related U.S. Application Data
(63) Continuation-in-part of application No. 13/620,141, filed on Sep. 14, 2012.
Application No. 13/620,141 is a continuation of application No. 13/403,923, filed on Feb. 23, 2012, now Pat. No. 8,309,127.
Application No. 13/403,923 is a continuation of application No. 13/285,981, filed on Oct. 31, 2011, now abandoned.
Application No. 13/285,981 is a continuation of application No. 12/324,808, filed on Nov. 26, 2008, now Pat. No. 8,067,033.
(60) Provisional application No. 60/991,628, filed on Nov. 30, 2007.

(51) Int. Cl. A61K 9/24; A61K 31/192; A61K 31/426

(52) U.S. Cl. CPC A61K 9/209; A61K 31/426; A61K 31/192
USPC 424/451; 514/370; 424/400; 424/464

(57) ABSTRACT

Stable pharmaceutical compositions of famotidine and ibuprofen in a single unit dosage form are disclosed herein. In some embodiments, the ibuprofen is in direct physical contact with the famotidine.

This application is a continuation-in-part of U.S. application Ser. No. 13/620,141, filed Sep. 14, 2012, which is a continuation of U.S. application Ser. No. 13/403,923, filed Feb. 23, 2012, now U.S. Pat. No. 8,309,127, which is a continuation of U.S. application Ser. No. 13/285,981, filed Oct. 31, 2011, which is a continuation of U.S. application Ser. No. 12/324,808, filed Nov. 26, 2008, now U.S. Pat. No. 8,067,033, which claims the benefit of U.S. Application No. 60/991,628, filed Nov. 30, 2007, each of which applications is incorporated by reference in its entirety.

Provided are pharmaceutical compositions containing ibuprofen and famotidine that may be useful in the field of medicine.

Ibuprofen, a non-steroidal anti-inflammatory drug (NSAID), has been used in humans for nearly forty years. While generally regarded as safe, ibuprofen and other NSAIDs can cause gastritis, dyspepsia, and gastric and duodenal ulceration. Gastric and duodenal ulceration is a consequence of impaired mucosal integrity resulting from ibuprofen-mediated inhibition of prostaglandin synthesis. This side-effect is a particular problem for individuals who take ibuprofen for extended periods of time, such as patients suffering from rheumatoid arthritis and osteoarthritis.

The risk of developing gastric or duodenal ulceration can be reduced by cotherapy with the drug famotidine. Famotidine blocks the action of the histamine type 2 (H2) receptor, leading to a reduction of acid secretion in the stomach. Reducing stomach acid with famotidine during treatment with certain nonsteroidal anti-inflammatory drugs is reported to decrease incidence of gastrointestinal ulcers (see Taha et al., 1996, “Famotidine for the prevention of gastric and duodenal ulcers caused by nonsteroidal anti-inflammatory drugs” N Engl J Med 334:1435-9, and Rostom et al., 2002, “Prevention of NSAID-induced gastrointestinal ulcers” Cochrane Database Syst Rev 4:CD002296).

Although NSAID plus famotidine cotherapy reduces risk of developing gastric or duodenal ulceration, such therapies are not widely used. One explanation for this observation is that patient compliance is more problematic with a regimen that requires administration of two separate dosage forms. Efforts to develop a single unit dosage form comprising both ibuprofen and famotidine have been successful (see U.S. application Ser. Nos. 11/489,275, filed Jul. 18, 2006, 11/489,705, filed Jul. 18, 2006, and 11/779,204, filed Jul. 17, 2007), but were made more challenging by the discovery that ibuprofen and famotidine are chemically incompatible. Moreover, those dosage forms that have been described could be improved with respect to stability under “forced degradation” or “accelerated” conditions of elevated temperature and humidity. Forced degradation conditions are intended to accelerate the process of chemical degradation for a period of time and are used to predict the effect of storage under more benign conditions (e.g., room temperature) for a longer period of time.

There remains a need for new and improved unit dosage forms comprising ibuprofen and famotidine that exhibit exceptional stability under forced degradation conditions. The present invention meets that need.

In one aspect, the present invention is directed to a pharmaceutical composition, comprising (i) a first portion comprising a therapeutically effective amount of famotidine, and (ii) a second portion comprising a therapeutically effective amount of ibuprofen in direct physical contact with the first portion, wherein the composition is stable for at least 1 month at 40° C. and 75% relative humidity.

In some cases, the first portion comprises famotidine in an amount from 24 mg to 28 mg, and the second portion comprises ibuprofen in an amount from 750 mg to 850 mg. In other cases, the first portion comprises about 26.6 mg of famotidine. In some embodiments, the first portion comprises famotidine in an amount from 24 mg to 28 mg, and the second portion comprises ibuprofen in an amount from 575 mg to 625 mg. In some embodiments, the first portion comprises famotidine in an amount from 12 mg to 14 mg, and the second portion comprises ibuprofen in an amount from 375 mg to 425 mg. In at least one embodiment, the ibuprofen is in the form of Ibuprofen DC 85™.

In some embodiments, the famotidine and the ibuprofen are in direct physical contact over a surface area that does not exceed 130 mm². In some embodiments, the surface area of direct physical contact between the famotidine and the ibuprofen does not exceed 120 mm². In some embodiments, the surface area of direct physical contact between the famotidine and the ibuprofen does not exceed 115 mm². In some embodiments, the surface area of direct physical contact between the famotidine and the ibuprofen does not exceed 100 mm². In at least one embodiment, the surface area of direct physical contact between the famotidine and the ibuprofen does not exceed 65 mm². In at least one embodiment, there is no direct physical contact between the famotidine and the ibuprofen.

### DETAILED DESCRIPTION

### I. Definitions

“Famotidine” refers to 3-[2-(diaminomethyleneamino)thiazol-4-ylmethylthio]-N-sulfamoylpropionamidine, as well as pharmaceutically acceptable salts thereof. Famotidine also is intended to encompass all known polymorphic forms, including without limitation the amorphous form, polymorphic Form A, Form B, or Form C and their mixtures. Famotidine can be prepared using art-known methods. Famotidine's properties have been described in the medical literature.

“Ibuprofen” refers to 2-(p-isobutylphenyl) propionic acid (C₁₃H₁₈O₂), including various polymorphic forms and pharmaceutically acceptable salts. Two enantiomers of ibuprofen exist. As used herein in the context of the pharmaceutical compositions described herein, “ibuprofen” refers to a racemic mixture of both enantiomers, as well as mixtures that contain more of one enantiomer than another (including, for example, mixtures enriched in the S-enantiomer), and enantiomerically pure preparations (including, for example, the S-enantiomer substantially free of the R-enantiomer). Ibuprofen is available commercially, typically as a racemic mixture, and, for example, ibuprofen with mean particle sizes of 25, 38, 50, or 90 microns can be obtained from BASF Aktiengesellschaft (Ludwigshafen, Germany). One ibuprofen product is a directly compressible formulation described in WO 2007/042445, a version of which is available from BASF under the trade name Ibuprofen DC 85. Ibuprofen DC 85 is a roller-compacted granulation comprising 85% ibuprofen, 6.6% microcrystalline cellulose, 5.4% colloidal silicon dioxide, and 2.9% croscarmellose sodium. Ibuprofen is also available from Albemarle Corporation and other companies. Ibuprofen's properties have been described in the medical literature.

A “therapeutically effective amount” of ibuprofen is an amount of ibuprofen or its pharmaceutically acceptable salt which eliminates, alleviates, or provides relief of the symptoms for which it is administered.

A “therapeutically effective amount” of famotidine is an amount of famotidine or its pharmaceutically acceptable salt which suppresses gastric acid secretion, or otherwise eliminates, alleviates, or provides relief of the symptoms for which it is administered.

An “excipient,” as used herein, is any component of an oral dosage form that is not an active pharmaceutical ingredient (i.e., ibuprofen and/or famotidine). Excipients include binders, lubricants, diluents, disintegrants, coatings, barrier layer components, glidants, and other components. Excipients are known in the art (see HANDBOOK OF PHARMACEUTICAL EXCIPIENTS, FIFTH EDITION, 2005, edited by Rowe et al., McGraw Hill). Some excipients serve multiple functions or are so-called high functionality excipients. For example, talc may act as a lubricant, and an anti-adherent, and a glidant. See Pifferi et al., 2005, “Quality and functionality of excipients” Farmaco. 54:1-14; and Zeleznik and Renak, Business Briefing: Pharmagenerics 2004.

As used herein, a “pharmaceutically acceptable” component is one that is suitable for use with humans and/or animals without undue adverse side effects (such as toxicity, irritation, and allergic response) commensurate with a reasonable benefit/risk ratio.

A “compartment” in the context of a unit dosage form is a physical region of a tablet or other dosage form. Two components of a unit dosage form are distinct compartments if there exists a recognizable demarcation between the two components, even though they may be in direct physical contact with one another.

The term “core,” as used herein, refers to a single interior compartment of a unit dosage form comprising famotidine.

The term “shell,” as used herein, refers to an exterior compartment of a unit dosage form comprising ibuprofen, which completely surrounds the core or famotidine compartment. As described herein, this exterior compartment may be over-coated for cosmetic or other reasons, in particular embodiments.

The term “barrier layer” refers to a layer or film that is interposed between the ibuprofen-containing compartment (e.g., an ibuprofen core or coated ibuprofen particles) and the famotidine or another H₂ receptor antagonist-containing compartment (e.g., famotidine-containing coating or coated famotidine particles). Materials useful in a “barrier layer” including in a “barrier layer coating” include, without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as, for example, hydroxymethylcellulose, hydroxyethylcellulose, and hydroxypropylcellulose; synthetic water-soluble cellulose-based lamina formers such as, for example, methyl cellulose and its hydroxyalkyl methylcellulose cellulose derivatives such as a member chosen from the group of hydroxyethyl methylcellulose, hydroxypropyl methyl cellulose, and hydroxybutyl methylcellulose; croscarmellose sodium; and other cellulose polymers such as sodium carboxymethylcellulose. Other lamina forming materials that can be used for this purpose include, for example, poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer.

The term “stable,” as used herein, refers to a composition in which the active pharmaceutical ingredients (i.e., ibuprofen and famotidine) are present in an amount of at least 90%, and preferably at least 95%, at least 96%, at least 97%, at least 98%, at least 99%, or at least 99.5% of the originally specified amount for each such ingredient, and no more than 3%, and preferably no more than 2%, no more than 1%, no more than 0.9%, no more than 0.8%, no more than 0.7%, or no more than 0.6% sulfamide is present after a specified period of time and under specified conditions.

The term “about,” as used herein, is intended to indicate a range (e.g., ±10%) caused by experimental uncertainty, variations resulting from manufacturing tolerances, or variations within the parameters of a label claim associated with a drug product.

The term “substantially,” as used herein with reference to the spherical or cylindrical shape of the core of a pharmaceutical composition or unit dosage form refers to variability resulting from manufacturing tolerances, as well as intentional deviations from these precise geometric shapes. For example, in a sphere the three axes are of identical length. In this context, the term “substantially” is intended to indicate a tolerance for a deviation of ±5% in the length of one or two of the axes in relation to the third axis, thus encompassing an oblongation or other variation of a spherical shape. In the context of a cylinder, the term “substantially” is intended to indicate a tolerance for a deviation of ±5% in the diameter of the cylinder along its length. For example, if the diameter increases from either end of the “cylinder” to a central position, the shape may be more appropriately referred to as a “barrel,” but is still intended to be encompassed by the phrase “substantially cylindrical in shape.”

A subject is “at elevated risk for developing an NSAID-induced ulcer” if the subject in more susceptible than the average individual to development of an ulcer when under treatment with an NSAID. A high odds ratio for risk of development of NSAID-associated ulcer complications is seen in individuals with a past complicated ulcer (odds ratio 13.5), individuals taking multiple NSAIDs or NSAIDs plus aspirin (odds ratio 9.0); individuals taking high doses of NSAIDs (odds ratio 7.0), individuals under anticoagulant therapy, such as low dose aspirin (odds ration 6.4), individuals with a past uncomplicated ulcer (odds ratio 6.1), and individuals older than 70 years (odds ratio 5.6) See, e.g., Gabriel et al., 1991, Ann Intern Med. 115:787; Garcia Rodriguez et al. 1994, Lancet 343:769; Silverstein et al. 1995, Ann Intern Med. 123:241; and Sorensen et al., 2000, Am J. Gastroenterol. 95:2218. Subjects at increased risk for developing an NSAID-induced ulcer may have one or more of these risk factors.

Subjects “at high risk for developing an NSAID-induced ulcer” are individuals older than 80 years of age and subjects with a history of NSAID-associated serious gastrointestinal complications (e.g., perforation of ulcers, gastric outlet obstruction due to ulcers, gastrointestinal bleeding).

### II. Pharmaceutical Compositions

Pharmaceutical compositions in accordance with the present invention comprise ibuprofen and famotidine in a single unit dosage form. In one aspect, the present invention relates to an oral dosage form comprising ibuprofen and famotidine, and optionally, one or more pharmaceutically acceptable excipients.

In one embodiment of the present invention, the pharmaceutical composition comprises a first compartment comprising a therapeutically effective amount of famotidine, and a second compartment comprising a therapeutically effective amount of ibuprofen in direct physical contact with the first compartment.

In other embodiments, a barrier layer is interposed between the two compartments. Generally, the barrier layer may comprise a water-soluble, pH independent film that promotes immediate disintegration for rapid release of the famotidine core. Materials that can be used for readily soluble films are well known in the art and include cellulose derivatives such as hydroxypropylmethyl cellulose, hydroxypropyl cellulose, hydroxypropyl methylcellulose phthalate, cellulose acetate phthalate, and ethyl cellulose; methacrylic polymers, amino-alkylmethacrylate copolymers (e.g. Eudragit™ E), polyvinyl acetate phthalate and polyvinyl alcohol (PVA). A plasticizer (e.g., triacetin, diethyl phthalate, tributyl sebacate or polyethylene glycol) may also be included. The barrier layer may include an anti-adherent or glidant (e.g., talc, fumed silica or magnesium stearate) and colorants such as titanium dioxide, iron oxide based colorants or others. In one embodiment the barrier layer comprises a non-toxic edible polymer, edible pigment particles, an edible polymer plasticizer, and a surfactant. Materials include, for example and not limitation, materials described in U.S. Pat. No. 4,543,370 (Colorcon), incorporated herein by reference. Exemplary barrier layers include OPADRY®, which is available from Colorcon (West Point Pa. USA); OPADRY II® which is available from Colorcon (West Point Pa. USA) and comprises HPMC, titanium dioxide, plasticizer and other components; and polyvinyl alcohol-polyethylene glycol copolymer marketed as Kollicoat® IR (BASF). Suitable barrier layers, for illustration and not limitation, include Kollicoat® IR (a polyvinyl alcohol-polyethylene glycol graft copolymer) and Kollicoat IR White® both manufactured by BASF Aktiengesellschaft (Ludwigshafen, Germany). The thickness of the barrier layer can vary over a wide range, but is generally in the range 20 to 3,000 microns, such as on the order of about 25 to 250 microns. Preferably the barrier layer retards the release of famotidine by less than 5 minutes, preferably less than 4 minutes and more preferably by less than 3 minutes.

### A. Famotidine Compartment

Pharmaceutical compositions in accordance with the present invention comprise a famotidine compartment structured as a first portion comprising a therapeutically effective amount of famotidine. The first portion can include both famotidine and, optionally, one or more pharmaceutically acceptable excipients.

The first portion can include an amount of famotidine suitable, for example, for the methods of treatment described hereinafter. For example, the first portion can comprise from 24 mg to 28 mg of famotidine, from 12 mg to 14 mg of famotidine, or the like, in various formulations consistent with the present invention. In some embodiments, the famotidine compartment comprises a first portion comprising about 13.3 mg or about 26.6 mg of famotidine.

### B. Ibuprofen Compartment

Pharmaceutical compositions of the present invention further comprise an ibuprofen compartment comprising a therapeutically effective amount of ibuprofen.

The ibuprofen compartment (i.e., the second portion) can include an amount of ibuprofen suitable, for example, for the methods of treatment described hereinafter, and, optionally, one or more pharmaceutically acceptable excipients. For example, the second portion can comprise from 750 mg to 850 mg of ibuprofen, from 575 mg to 625 mg of ibuprofen, from 375 mg to 425 mg of ibuprofen, or the like, in various formulations consistent with the present invention. In some embodiments, the second portion comprises a surrounding portion comprising from 775 mg to 825 mg of ibuprofen, or, in one embodiment, 800 mg of ibuprofen. In other embodiments, the compositions and/or unit dosage forms of the present invention comprise ibuprofen and famotidine in a ratio of from about 29:1 to about 31:1, and preferably in a ratio of about 30:1. In some embodiments, the ibuprofen is in the form of Ibuprofen DC 85™.

### C. Excipients

A variety of excipients may be combined with famotidine and/or ibuprofen in their respective compartments of the pharmaceutical compositions of the present invention. As mentioned above, the provision of various excipients may be useful to impart particular qualities to either the famotidine component or the ibuprofen component of the pharmaceutical composition, or to provide a beneficial characteristic that may be desirable for processing to prepare the tablet-in-tablet formulation. Pharmaceutically acceptable excipients useful in compositions of the present invention can include binders, lubricants, diluents, disintegrants, and glidants, or the like, as known in the art. See e.g., HANDBOOK OF PHARMACEUTICAL MANUFACTURING FORMULATIONS, 2004, Ed. Sarfaraz K Niazi, CRC Press; HANDBOOK OF PHARMACEUTICAL ADDITIVES, SECOND EDITION, 2002, compiled by Michael and Irene Ash, Synapse Books; and REMINGTON SCIENCE AND PRACTICE OF PHARMACY, 2005, David B. Troy (Editor), Lippincott Williams & Wilkins.

Binders useful in compositions of the present invention are those excipients that impart cohesive qualities to components of a pharmaceutical composition. Commonly used binders include, for example, starch; sugars, such as, sucrose, glucose, dextrose, and lactose; cellulose derivatives such as powdered cellulose, microcrystalline cellulose, silicified microcrystalline cellulose (SMCC), hydroxypropylcellulose, low-substituted hydroxypropylcellulose, hypromellose (hydroxypropylmethylcellulose); and mixtures of these and similar ingredients.

Lubricants can be added to components of the present compositions to reduce sticking by a solid formulation to the equipment used for production of a unit does form, such as, for example, the punches of a tablet press. Examples of lubricants include magnesium stearate and calcium stearate. Other lubricants include, but are not limited to, aluminum-stearate, talc, sodium benzoate, glyceryl mono fatty acid (e.g., glyceryl monostearate from Danisco, UK), glyceryl dibehenate (e.g., CompritolATO888™ Gattefosse France), glyceryl palmito-stearic ester (e.g., Precirol™ Gattefosse France), polyoxyethylene glycol (PEG, BASF) such as PEG 4000-8000, hydrogenated cotton seed oil or castor seed oil (Cutina H R, Henkel) and others.

Diluents can be added to components of a pharmaceutical composition to increase bulk weight of the material to be formulated, e.g. tabletted, in order to achieve the desired weight.

Disintegrants useful in the present compositions are those excipients included in a pharmaceutical composition in order to ensure that the composition has an acceptable disintegration rate in an environment of use. Examples of disintegrants include starch derivatives (e.g., sodium carboxymethyl starch and pregelatinized corn starch such as starch 1500 from Colorcon) and salts of carboxymethylcellulose (e.g., sodium carboxymethylcellulose), crospovidone (cross-linked PVP polyvinylpyrrolidinone (PVP), e.g., Polyplasdone™ from ISP or Kollidon™ from BASF).

Glidants refer to excipients included in a pharmaceutical composition to keep the component powder flowing as a tablet is being made, preventing formation of lumps. Nonlimiting examples of glidants are colloidal silicon dioxides such as CAB-O-SIL™ (Cabot Corp.), SYLOID™, (W.R. Grace & Co.), AEROSIL™ (Degussa), talc, and corn starch.

### D. Stability of Compositions

Compositions of the present invention comprising a famotidine compartment and an ibuprofen compartment are stable for extended periods under “forced degradation” conditions of elevated temperature and relative humidity. For example, compositions of famotidine and ibuprofen prepared as described in the “Examples” section, hereinbelow, exhibit unexpectedly dramatic improvements in stability at 40° C. and 75% relative humidity, relative to alternative designs (e.g., barrier-coated famotidine multiparticulates in a matrix comprising ibuprofen). Moreover, using the design of the present invention, the barrier layer can be omitted without sacrificing stability.

“Forced degradation” conditions (e.g., 40° C. and 75% relative humidity) are used to evaluate the long-term storage stability of a pharmaceutical ingredient or composition. In general terms, a stable composition is one which comprises the pharmaceutically active ingredients in an amount, for example 95%, relative to the amount initially present in the particular composition. Stability may be determined, using forced degradation or other methods, for periods of 1 week, 1 month, 2 months, 3 months, 4 months, 5 months, 6 months, 9 months, 12 months, 15 months, 18 months, 24 months, 30 months, 36 months, longer.

Stability may also be determined by the presence and quantity of impurities. A principal degradant produced through the chemical interaction of famotidine and ibuprofen in compositions of the present invention is sulfamide, i.e., the sulfamide degradation product of famotidine, namely, 3-[2-(diaminomethyleneamino)-1,3-thiazol-4-ylmethylthio-N-sulfamoyl-propanamide. A quantitative determination of the presence of sulfamide in a unit dose form of the present invention held under forced degradation conditions for a period of time yields valuable information about the long-term stability of the composition under ordinary (e.g., room temperature) storage conditions.

Assays for evaluating the stability of a pharmaceutical composition, such as those described in the present invention, are known in the pharmaceutical arts. For example, one can determine the percentage of active pharmaceutical ingredients present in a given composition, as well as the presence and percentage of impurities, through the use of standard analytical techniques.

### III. Methods of Making Compositions

It is within the ability of one of ordinary skill in the art, guided by the present disclosure and with reference to the pharmaceutical literature, to prepare and manufacture unit dosage forms of the invention in accordance with the methods of the invention.

In some embodiments, the pharmaceutical composition is in a tablet form, including without limitation, a core-shell architecture, a bilayer tablet, or a trilayer tablet. In some embodiments, the pharmaceutical composition is in the form of a scored tablet.

In some embodiments, the pharmaceutical composition is in a tablet form, including without limitation, a core-shell architecture, a bilayer tablet, or a trilayer tablet. In some embodiments, the pharmaceutical composition is in the form of a scored tablet.

In one embodiment, the unit dosage form comprises a tablet dosage form having a first compartment comprising famotidine and a second compartment comprising ibuprofen.

In some embodiments, the pharmaceutical composition has a trilayer architecture. Accordingly, also provided is a pharmaceutical composition wherein the first compartment corresponds to a first layer of the pharmaceutical composition, a portion of the second compartment corresponds to a second layer of the pharmaceutical composition adjacent to a first side of said first layer, and the remainder of the second compartment corresponds to a third layer of the pharmaceutical composition adjacent to a second side of the first layer, where the first layer and the second layer are separated by a first barrier layer, and the first layer and the third layer are separated by a second barrier layer. Also provided is a pharmaceutical composition comprising a first layer comprising

a first layer comprising a therapeutically effective amount of an H₂ receptor antagonist,

a second layer comprising ibuprofen, and

a third layer comprising ibuprofen,

wherein the first layer is adjacent to a first side of the first layer, and the third layer is adjacent to a second side of the first layer,

wherein the total amount of ibuprofen in the pharmaceutical composition is a therapeutically effective amount, and

wherein the first layer and the second layer are separated by a first barrier layer, and the first layer and the third layer are separated by a second barrier layer.

In some embodiments, the first barrier layer is the same, both in amount and content, as the second barrier layer. In some embodiments, the first barrier layer is different, either in amount and/or content, from the second barrier layer.

Optionally, the tablet is coated by one or more over-coating layers, for example, to improve appearance, taste, swallowability, or for other reasons. In another embodiment, a barrier layer is interposed between the famotidine compartment and the ibuprofen compartment. Methods for formulation and manufacture of pharmaceutical unit dose forms are known in the art, see, e.g., HANDBOOK OF PHARMACEUTCAL MANUFACTURING FORMULATIONS, 2004, Ed. Sarfaraz K Niazi, CRC Press; HANDBOOK OF PHARMACEUTCAL ADDITIVES, SECOND EDITION, 2002, compiled by Michael and Irene Ash, Synapse Books; and REMINGTON SCIENCE AND PRACTICE OF PHARMACY, 2005, David B. Troy (Editor), Lippincott Williams & Wilkins. One of ordinary skill in the art guided by this disclosure will be able to make a variety of suitable oral unit dose forms.

In general, a tablet-in-tablet composition is produced by first preparing a tablet “core” from a first component, and then applying a “shell” (e.g., through compression, or the like) of a second component in a manner such that the finished formulation comprises the core surrounded by the shell. In embodiments in which a barrier layer is interposed between the famotidine core and the ibuprofen shell, the barrier may be applied to the “core” by, e.g., spray coating, or the like.

As noted above, in some embodiments, the tablets are coated for oral administration to make the tablet easier to swallow, to mask taste, for cosmetic reasons, or for other reasons. Coating of tablets and caplets is well known in the art. Coating systems are typically mixtures of polymers, plasticisers, coloring agents and other excipients, which can be stirred into water or an organic solvent to produce a dispersion for the film coating of solid oral dosage forms such as tablets. Often, a readily soluble film is used. Materials that can be used for readily soluble films include cellulose derivatives (such as hydroxypropylmethyl cellulose) or amino-alkylmethacrylate copolymers (e.g. Eudragit™ E). Suitable coat layers, for illustration and not limitation, include Kollicoat® IR (a polyvinyl alcohol-polyethylene glycol graft copolymer) and Kollicoat IR White®, both manufactured by BASF Aktiengesellschaft (Ludwigshafen, Germany). In some embodiments, the coating agent comprises OPADRY II blue.

In some embodiments, the tablet is a sublingual or buccal tablet.

In some embodiments, the pharmaceutical composition is in the form of a soft gel capsule.

In some embodiments, the soft gel capsule comprises gelatin. In some embodiments, the soft gel capsule comprises gelatin, water, an opacifier, and a plasticizer, such as glycerin and/or sorbitol(s). In some embodiments, the soft gel capsule is commercially available from Catalent Pharma Solutions.

In some embodiments, the pharmaceutical composition is in the form of a hard gel capsule.

In some embodiments, a first compartment in the capsule further comprises a nonaqueous liquid such as an oil wherein the famotidine is dissolved or suspended in the nonaqueous liquid. In some embodiments, a second compartment in the capsule further comprises a nonaqueous liquid such as an oil wherein the ibuprofen is dissolved or suspended in the nonaqueous liquid.

In some embodiments, the capsule is filled with barrier-coated granules comprising famotidine that are blended with ibuprofen, such as in the form of Ibuprofen DC 85™ optionally with one or more excipients. In some embodiments, the capsule is filled with granules comprising famotidine that are blended with ibuprofen granules, wherein each type of granules is coated with a barrier layer. In some embodiments, the same type of barrier layer is used; in some embodiments, the barrier layer surrounding the famotidine granules is different from the barrier layer surrounding the ibuprofen granules.

In some embodiments, a first compartment in the capsule comprises a core of famotidne. In some embodiments, the famotidne is present as multiple particles. In some embodiments, the famotidne is present as multiple particles and are blended or otherwise mixed with powdered ibuprofen powder, optionally with one or more excipients. In some embodiments, the famotidne is present as multiple particles and are blended or otherwise mixed with ibuprofen granules or particles.

In some embodiments, the pharmaceutical composition is in a chewable form. Accordingly, also provided is a pharmaceutical composition comprising:

a first compartment comprising a therapeutically effective amount of famotidine, and

a second compartment comprising a therapeutically effective amount of ibuprofen, and

further comprising a binding agent and a sweetener,

wherein the first compartment is separated from the second compartment, and wherein the pharmaceutical composition is in a chewable form.

In some embodiments, the binding agent is chosen from pectin, gelatin, starch, and mixtures thereof. Chewable forms for delivering pharmaceutical agents and methods for making such forms are well know in the art. See, e.g., US Patent Publication No. 2010/0330058 which is incorporated herein by reference.

In some embodiments, the pharmaceutical composition is in a form that dissolves and/or disintegrates orally. Accordingly, also provided is a pharmaceutical composition comprising:

a first compartment comprising a therapeutically effective amount of famotidine, and

a second compartment comprising a therapeutically effective amount of ibuprofen,

wherein the first compartment is separated from the second compartment, and

wherein the first compartment and the second compartment are present in an orally dissolving film.

In some embodiments, the pharmaceutically composition comprises a single layer of orally dissolvable film. In some embodiments, the pharmaceutically composition comprises multiple layers of orally dissolvable film. In some embodiments, pharmaceutically composition comprises a first layer of orally dissolvable film comprising a therapeutically effective amount of ibuprofen and a second layer of orally dissolvable film comprising a therapeutically effective amount of famotidine. Orally dissolving films and methods for making such films are well know in the art. See, e.g., US Patent Publication No. 2010/0227854 which is incorporated herein by reference.

In some embodiments, the pharmaceutically composition comprises a powder in which the famotidine and the ibuprofen are mixed with other powdered excipients to produce a final product for oral administration. In some embodiments, the powder is dissolved in a liquid, e.g., water or juice, before administration. In some embodiments, the famotidine is present in the powder as famotidine granules that are coated with a barrier layer. In some embodiments, the ibuprofen is present in the powder as ibuprofen granules that are coated with a barrier layer. In some embodiments, both the ibuprofen and the famotidine are coated with a barrier layer, wherein the barrier layers used for each API are the same or different.

In some embodiments, the famotidine powder is separated from the ibuprofen powder, e.g., in different vials or containers, and the powders are administered to the subject at about the same time. In some embodiments, the famotidine powder is mixed with the ibuprofen powder prior to administration.

In some embodiments, the pharmaceutical composition is effervescent and further comprises sodium bicarbonate (or another means for generating carbon dioxide) and an organic acid, such as citric acid or tartaric acid (or another means to induce generation of the carbon dioxide.)

### IV. Methods of Treatment

In one aspect, the present invention is directed to methods of treating subjects in need of ibuprofen and famotidine treatment. Methods applicable to the present invention are described in U.S. Pat. No. 8,067,451, and incorporated herein by reference. Subjects in need of ibuprofen and famotidine treatment include those individuals at elevated risk for developing an NSAID-induced ulcer (i.e., the subject is more susceptible than the average individual to development of an ulcer when under treatment with an NSAID). More generally, subjects in need of ibuprofen and famotidine treatment are those individuals who receive a therapeutic benefit from administration of ibuprofen and famotidine.

Ibuprofen is indicated for treatment of mild to moderate pain, dysmenorrhea, inflammation, and arthritis. In one embodiment, the subject in need of ibuprofen treatment with a dosage form of the invention is under treatment for a chronic condition. For example and without limitation, a subject in need of ibuprofen treatment may be an individual with rheumatoid arthritis, an individual with osteoarthritis, an individual suffering from chronic pain (e.g., chronic low back pain, chronic regional pain syndrome, chronic soft tissue pain), or an individual suffering from a chronic inflammatory condition. In general, a subject under treatment for a chronic condition requires ibuprofen treatment for an extended period, such as at least one month, at least four months, at least six months, or at least one year, and at least some of these subjects can benefit from receiving famotidine in combination with ibuprofen during such treatment period. In another embodiment, the subject in need of ibuprofen treatment is under treatment for a condition that is not chronic, such as acute pain, dysmenorrhea or acute inflammation and can benefit from receiving famotidine in combination with ibuprofen during such treatment.

In certain embodiments oral dosage forms of the invention are formulated so that release of both active pharmaceutical ingredients (APIs) occurs (or begins to occur) at about the same time. “At about the same time” means that release of one API begins within 5 minutes of the beginning of release of the second API, sometimes with 4 minutes, sometimes within 3 minutes, sometimes within 2 minutes, and sometimes essentially simultaneously. “At about the same time” can also mean that release of one API begins before release of the second API is completed. That is, the dosage form is not designed so that one of the APIs is released significantly later than the other API. To achieve this, combinations of excipients (which may include one or more of a binder, a lubricant, a diluent, a disintegrant, a glidant and other components) are selected that do not substantially retard release of an API. See e.g., HANDBOOK OF PHARMACEUTICAL MANUFACTURING FORMULATIONS, 2004, Ed. Sarfaraz K Niazi, CRC Press; HANDBOOK OF PHARMACEUTICAL ADDITIVES, SECOND EDITION, 2002, compiled by Michael and Irene Ash, Synapse Books; and REMINGTON SCIENCE AND PRACTICE OF PHARMACY, 2005, David B. Troy (Editor), Lippincott Williams & Wilkins.

In the unit dose forms of the invention, both the famotidine or ibuprofen are formulated for immediate release, and not for release profiles commonly referred to as delayed release, sustained release, or controlled release. For example, in one embodiment, the unit dosage form is formulated so that famotidine and ibuprofen are released rapidly under neutral pH conditions (e.g., an aqueous solution at about pH 6.8 to about pH 7.4, e.g., pH 7.2). In this context, “rapidly” means that both APIs are significantly released into solution within 20 minutes under in vitro assay conditions. In some embodiments both APIs are significantly released into solution within 15 minutes under in vitro assay conditions. In this context, “significantly released” means that at least about 60% of the weight of the API in the unit dosage form is dissolved, or at least about 75%, or at least about 80%, or at least about 90%, and sometimes at least about 95%. In one embodiment, both famotidine and ibuprofen are at least 95% released in 30 minutes.

Dissolution rates may be determined using known methods. Generally an in vitro dissolution assay is carried out by placing the famotidine-ibuprofen unit dosage form(s) (e.g., tablet(s)) in a known volume of dissolution medium in a container with a suitable stirring device. Samples of the medium are withdrawn at various times and analyzed for dissolved active substance to determine the rate of dissolution. Dissolution may be measured, for example, as described for ibuprofen in the USP or, alternatively, as described for famotidine in the USP. Briefly, in this exemplary method, the unit dose form (e.g., tablet) is placed in a vessel of a United States Pharmacopeia dissolution apparatus II (Paddles) containing 900 ml dissolution medium at 37° C. The paddle speed is 50 RPM. Independent measurements are made for at least three (3) tablets. In one suitable in vitro assay, dissolution is measured using a neutral dissolution medium such as 50 mM potassium phosphate buffer, pH 7.2 (“neutral conditions”).

Alternatively, dissolution rates may be determined under low pH conditions. Release under low pH conditions can be measured using the in vitro dissolution assay described above, but using, for example, 50 mM potassium phosphate buffer, pH 4.5, as a dissolution medium. As used in this context, the APIs are released rapidly at low pH when a substantial amount of both APIs is released into solution within 60 minutes under low pH assay conditions. In some embodiments, a substantial amount of both APIs is released into solution within 40 minutes under low pH assay conditions. In some embodiments, a substantial amount of both APIs is released into solution within 20 minutes under low pH assay conditions. In some embodiments, a substantial amount of both APIs is released into solution within 10 minutes under low pH assay conditions. In this context, a “substantial amount” means at least 15%, or at least 20%, or at least 25% of ibuprofen is dissolved and at least 80%, or at least 85%, or at least 90% of famotidine is dissolved.

In some cases, dosage forms of the present invention are designed for three times per day (TID) administration of famotidine and ibuprofen to a patient in need thereof.

When administered to avoid or mitigate the ulcerogenic effects of long-term NSAID therapy, famotidine has been administered at 40 mg BID (see Taha et al., 1996, supra). However, as described in co-pending application Ser. Nos. 11/489,275 and 11/489,705, both filed Jul. 18, 2006 and incorporated herein by reference, it has now been determined using pharmacokinetic modeling and in clinical trials, that TID administration of famotidine provides a therapeutic effect superior to that achieved by BID dosing. For example, on average, TID administration of famotidine results in intragastric pH higher than 3.5 for a greater proportion of the dosing cycle than conventional BID dosing.

Treatment using the methods of TID administration also results in reduced interpatient variability with respect to gastric pH in a population of patients receiving an ibuprofen-famotidine combination treatment. This reduction increases predictability of the treatment and reduces the likelihood that any particular patient will experience detrimental gastric pH in the course of ibuprofen-famotidine combination therapy.

Thus, in another aspect, the present invention provides a method for administration of ibuprofen to a patient in need of ibuprofen treatment by administering an oral dosage form comprising a therapeutically effective amount of ibuprofen and a therapeutically effective amount of famotidine, wherein the oral dosage form comprises a tablet-in-tablet formulation for administration three times per day (TID).

### EXAMPLES

The following examples are offered to illustrate, but not to limit, the claimed invention.

### Example 1

A tablet-in-tablet composition of famotidine and ibuprofen according to the present invention can be prepared by first preparing a famotidine core, which is then surrounded by an ibuprofen shell and an optional over-coating. The famotidine core is prepared by (i) combining 26.6 mg famotidine, 10.0 mg lactose monohydrate, 34.6 mg microcrystalline cellulose, 4.0 mg croscarmellose sodium, and 0.4 mg colloidal silicon dioxide in a suitably sized V-blender; (ii) mixing the combined ingredients for approximately ten minutes; (iii) discharging the blended materials from the blender and passing them through a #20 mesh screen; (iv) transferring the screened material back into the V-blender and mixing for approximately ten additional minutes; (vi) passing 1.2 mg magnesium stearate through a #30 mesh screen; (vii) adding the screened magnesium stearate to the blended material in the V-blender and mixing for approximately three additional minutes; (viii) discharging the blended material into a polyethylene lined container; and (ix) compressing the blended material into a tablet (i.e., a famotidine core) on a rotary tablet press using 0.2187″ plain round SC (standard concave round) tooling. The famotidine core is then centered in a tablet-in-tablet composition by compressing 941.2 mg of Ibuprofen DC 85™. (comprises 800 mg of ibuprofen) around the famotidine core using a tablet press and 0.4100″.times.0.7500″ oval plain tooling. The tablet-in-tablet is then preferably over-coated by placement in a suitably sized perforated coating pan to which a dispersion of Opadry II (Colorcon, Inc., West Point, Pa.) in water is added to coat the tablet-in-tablet to a weight gain of 3%.

A summary of the materials used in the tablet-in-tablet composition described in Example 1 are provided in the table below.

| | | Amount | Amount | |
|---|---|---|---|---|
| Item | Material | (% w/w) | (mg) | Function |
| 1 | Famotidine | 2.54 | 26.6 | API |
| 2 | Lactose monohydrate | 0.95 | 10.0 | Binder |
| | (DCL 21) | | | |
| 3 | Microcrystalline cellulose | 3.30 | 34.6 | Binder |
| | (Avicel PH102) | | | |
| 4 | Croscarmellose sodium | 0.38 | 4.0 | Disintegrant |
| | (Ac-di-sol) | | | |
| 5 | Colloidal silicon dioxide | 0.04 | 0.4 | Glidant |
| | (Cab-o-sil M5P) | | | |
| 6 | Magnesium stearate | 0.11 | 1.2 | Lubricant |
| 7 | Ibuprofen granules (DC-85)* | 89.75 | 941.2 | API |
| 8 | Opadry II (85F18422 White) | 2.93 | 30.7 | Over-coat |
| 9 | Purified Water | — | q.s. | Process aid |
| Total | — | 100.00 | 1048.7 | |

*Contains 800 mg of ibuprofen.

### Example 2

A tablet-in-tablet composition of famotidine and ibuprofen in accordance with the present invention, and which includes a barrier layer interposed between the active pharmaceutical ingredients can be prepared as described in Example 1, with the following modification. Following preparation of the famotidine core by compressing the blended material into a tablet (i.e., step (ix)), the tablet core is coated with a barrier layer by placement in a suitably sized perforated coating pan to which a dispersion of Opadry (YS-1-7003) (Colorcon) in water is added to coat the tablet core to a weight gain of 5%. With reference to the materials identified in the table above, a weight gain of 5% requires about 3.8 mg of Opadry.

### Example 3

Stability of three distinct famotidine plus ibuprofen formulations was evaluated under “forced degradation” conditions of 40° C. and 75% relative humidity to assess the viability of the different combinations of the active pharmaceutical ingredients. Surprisingly, a tablet-in-tablet formulation in accordance with the present invention exhibited remarkably improved stability, as shown in the table below, as compared to both a multiparticulate formulation and a bilayer formulation, each of which relies on the presence of a barrier between the famotidine and ibuprofen to reduce chemical interaction and degradation of the active pharmaceutical ingredients.

The multiparticulate formulation comprises an ibuprofen matrix into which are dispersed a plurality of famotidine beads. Each famotidine bead consists of a microcrystalline cellulose core surrounded by a layer of famotidine which is coated with a protective barrier layer (e.g., Opadry). A description of the process of making such beads is provided in Example 9 of U.S. Pat. No. 8,067,451, which is incorporated herein by reference for all purposes. The bilayer tablet formulation similarly comprises a layer of famotidine beads sandwiched together with a layer of ibuprofen.

1 Month Stability of Famotidine + Ibuprofen Compositions
(@ 40° C. and 75% Relative Humidity)

| | | | Tablet-in- | |
|---|---|---|---|---|
| | | | Tablet | |
| | | | Formulation | Tablet-in-Tablet |
| Stability | Multiparticulate | Bilayer | (Direct | Formulation |
| Indicator | Formulation | Formulation | Contact)^{†} | (Barrier Coated)^{††} |
| % Sulfamide | 3.55 | 0.91 | 0.56 | 0.00 |
| Ibuprofen | 0.23 | 2.01 | 0.00 | 0.00 |
| Impurities** | | | | |
| Total | 4.90 | 3.00 | 0.70 | 0.00 |
| Impurities | | | | |
| % Ibuprofen* | 100.3 | 100.5 | 99.5 | 100.8 |
| % | 95.5 | 103.2 | 94.6 | 96.7 |
| Famotidine* | | | | |

*Calculated from initial sample assessment; each formulation includes 26.6 mg famotidine and 800 mg ibuprofen.
**Ibuprofen impurities comprise components attributable to the degradation of ibuprofen.
^{†}Prepared according to the procedure described in Example 1.
^{††}prepared according to the procedure described in Example 2.

As shown in the table above, the tablet-in-tablet formulation in accordance with the present invention shows a markedly improved stability profile, as compared with the multiparticulate and bilayer formulations of the same chemically incompatible active ingredients, in terms of both the presence of sulfamide, the principal famotidine degradant, as well as total impurities. In the multiparticulate formulation, the issue of chemical incompatibility is addressed by the barrier layer surrounding each famotidine bead dispersed throughout the ibuprofen matrix. Similarly, in the bilayer formulation, barrier-coated famotidine beads make up the famotidine layer of the bilayer construction.

### Example 4

### Manufacture of Ibuprofen/Famotidine Unit Dose Forms

### (1) Formulation (A)

This example describes manufacture of a tablet containing ibuprofen granules and coated famotidine granules. See, also, PCT/US2011/038622, filed May 31, 2011, which is incorporated by reference for all purposes.

a. Ibuprofen Granules

| | | | | % of |
|---|---|---|---|---|
| | | | Amount per | Tablet |
| Item | Component | Function | Tablet (mg) | (w/w) |
| 1 | Ibuprofen, USP | Active | 800.0 | 58.49 |
| 2 | Lactose | Binder | 300.0 | 21.93 |
| | monohydrate, NF | | | |
| 3 | Colloidal | Glidant | 6.0 | 0.44 |
| | silicon dioxide, NF | | | |
| 4 | Croscarmellose | Disintegrant | 30.0 | 2.19 |
| | sodium, NF | | | |
| 5 | Hypromellose, USP | Filler | 24.0 | 1.75 |
| 6 | Purified Water, USP | Solvent | * | * |

| | | |
|---|---|---|
| Total weight | 1160.0 | 84.80 |

* Water is removed during the process and therefore not factored in tablet weight.

Items 1-5 are sifted through Quadro Comil 16-mesh and mixed (Blend 1). Item 5B is dissolved in water and slowly added to Blend 1 using a mixer. Additional water is added and mixed. The wet material is dried at 50° C. for 12 h, milled using a 16-mesh screen with appropriate spacer, and dried until the LOD at 50° C. is below 0.5% w/w. Dried granules and extra granular material is transferred to a V-blender and mixed for 3 minutes.

b. Famotidine Granules

| | | | | |
|---|---|---|---|---|
| | | | | % of |
| | | | Amount per | Tablet |
| Item | Component | Function | Tablet (mg) | (w/w) |

Famotidine Granules

| | | | | |
|---|---|---|---|---|
| 1 | Famotidine, USP | Active | 26.6 | 1.94 |
| 2 | Opadry II White | Coating agent | 7.1 | 0.52 |
| | (Y-22-7719) | | | |
| 3 | Talc, Imperial, USP | Thickening agent | 1.8 | 0.13 |
| 4 | Microcrystalline | Binder | 35.5 | 2.60 |
| | cellulose, NF | | | |
| 5 | Purified Water, | Solvent | * | * |
| | USP** | | | |

| | | |
|---|---|---|
| Total weight | 71.0 | 5.19 |

Barrier Coating

| | | | | |
|---|---|---|---|---|
| 6 | Opadry White | Coating agent | 7.1 | 0.52 |
| | (YS-1-7003) | | | |
| 7 | Purified Water, | Solvent | * | * |
| | USP*** | | | |

| | | |
|---|---|---|
| Total weight | 7.1 | 0.52 |

* Water is removed during the process and therefore not factored in tablet weight.
**Famotidine suspension is 20% solids w/w.
***Protective coating suspension is 13% solids w/w.

Set up the Glatt fluid bed processor and add microcrystalline cellulose to Glatt. Disperse famotidine in purified water under mechanical stirring for 5 minutes. Add Opadry followed by talc and let it run for 30 minutes. Homogenize the above suspension for 20-30 minutes. Keep mixing at slow speed to avoid air entrapment.

Set up the peristaltic pump and spray the drug suspension completely. Dry the product to a product temperature of around 40-44° C. Sift the spray granulated famotidine through Quadro comil #20 mesh.

Spray Opadry suspension equivalent to 10% weight gain in the Glatt fluid bed processor. Dry the final product to a product temperature of around 40-44° C. Discharge and sift it through ASTM #30 mesh to remove any agglomerate.

c. Final Blending

| | | | | % of |
|---|---|---|---|---|
| | | | Amount per | Tablet |
| Item | Component | Function | Tablet (mg) | (w/w) |
| 1 | Ibuprofen granules | In-process | 1160.0 | |
| | | granules | | |
| 2 | Famotidine granules | In-process | 78.1 | |
| | | granules | | |
| 3 | Colloidal silicon dioxide, | Glidant | 4.0 | 0.29 |
| | NF | | | |
| 4 | Croscarmellose sodium, NF | Disintegrant | 30.0 | 2.19 |
| 5 | Silicified microcrystalline | Binder | 47.0 | 3.44 |
| | cellulose (Prosolv | | | |
| | SMCC 90) | | | |
| 6 | Magnesium stearate, NF | Lubricant | 9.0 | 0.66 |

| | | |
|---|---|---|
| Total Tablet Weight | 1328.1 | 6.58 |

Weight appropriate amount of ibuprofen granules, famotidine granules and the extra-granular materials. Blend geometrically famotidine and ibuprofen granules in appropriate blenders.

Add the sifted extra-granular materials (Prosolv SMCC 90, croscarmellose sodium and colloidal silicon dioxide sifted through 16-mesh screen) to above granules and mix for 3 minutes.

Sift magnesium stearate through 30 mesh screen and transfer to the above blender and lubricate for 3 minutes.

d. Tabletting

Set DC-16 compression machine with bisect punches and compress the blend to tablets with target weight of 1.328 g, hardness of 10-20 Kp, disintegration time less than 15 minutes.

e. Film Coating

| | | | | % of |
|---|---|---|---|---|
| | | | Amount per | Tablet |
| Item | Component | Function | Tablet (mg) | (w/w) |
| 1 | Ibuprofen/Famotidine core | In-process | 1328.1 | |
| | tablets | granules | | |
| 2 | Opadry II White | Film Coating | 39.9 | 2.91 |
| | (85F18422) | | | |
| 2 | Purified Water, USP | Solvent | * | * |

| | | |
|---|---|---|
| Total weight | 1368.0 | 100% |

* Water is removed during the process and therefore not factored in tablet weight.

Disperse Opadry II white (85 F18422) in water under mechanical stirring. Continue mixing for 45 minutes at slow speed. Load approximately 80-90 kg of compressed tablets in Acella Cota with a 48″ coating pan. Coat the tablets to a weight gain of 2.5-3.5% w/w following optimum coating parameters.

In other related embodiments tablets are made as above except that the amount of any non-API component can vary from the amounts above by up to plus or minus 10%. For example, the lactose monohydrate component could vary in the range from about 23.3 to about 28.4%. APIs can vary in amounts as described elsewhere herein.

f. Stability of Ibuprofen/Famotidine Tablet

Tablets were prepared as described above. The stability profile of the tablet is provided below.

| | | Ibuprofen | | | | |
|---|---|---|---|---|---|---|
| | Time | | 4- | Famotidine | | Total |
| Conditions | Point | Assay | IBAP¹ | Assay | Sulfamide | Impurities |
| 50° C. | 1 week | 98.4 | 0.0 | 67.8 | 21.7 | 32.0 |
| 40° C./ | 2 weeks | 98.6 | 0.06 | 103.1 | 0.87 | 2.0 |
| 75% RH | 1 month | 98.6 | 0.0 | 99.9 | 3.2 | 4.4 |
| 25° C./ | 1 month | 99.6 | 0.0 | 105.1 | 0.1 | 0.3 |
| 60% RH | | | | | | |

### (2) Formulation (B): Tablet-in-Tablet

This example describes manufacture of a tablet containing ibuprofen granules and coated famotidine cores.

A tablet-in-tablet composition of famotidine and ibuprofen described herein can be prepared by first preparing a famotidine core, which is then coated with a barrier layer, then surrounded by an ibuprofen shell and an optional over-coating. The famotidine core is prepared by (i) combining 26.6 mg famotidine, 12.7 mg lactose, anhydrous, 44.1 mg microcrystalline cellulose, 4.7 mg croscarmellose sodium, and 0.7 mg colloidal silicon dioxide in a suitably sized V-blender; (ii) mixing the combined ingredients for approximately ten minutes; (iii) discharging the blended materials from the blender and passing them through a #20 mesh screen; (iv) transferring the screened material back into the V-blender and mixing for approximately ten additional minutes; (vi) passing 1.4 mg magnesium stearate through a #30 mesh screen; (vii) adding the screened magnesium stearate to the blended material in the V-blender and mixing for approximately three additional minutes; (viii) discharging the blended material into a polyethylene lined container; and (ix) compressing the blended material into a tablet (i.e., a famotidine core) on a rotary tablet press using 0.2187″ plain round SC tooling.

The famotidine tablet core is coated with a barrier layer by placement in a suitably sized perforated coating pan to which a dispersion of Opadry (YS-1-7003) (Colorcon) in water is added to coat the tablet core to a weight gain of 5.5%, which results in about 5 mg of added solids after drying.

After the barrier layer has dried, 941.2 mg of Ibuprofen DC 85 (containing 800 mg of ibuprofen) is blended with 111.1 mg microcrystalline cellulose, 55.5 mg povidone, and 2.8 mg magnesium stearate, then granulated. The granulated ibuprofen mixture is then compressed around the barrier-coated famotidine core using a tablet press. Finally, the tablet is over-coated by placement in a suitably sized perforated coating pan containing a dispersion of Opadry II blue (85F99093) (Colorcon) in water to coat the tablet to a weight gain of 4.0% (48.2 mg added solids after drying).

The composition of the tablet-in-tablet formulation (B) is provided below.

| | | | | |
|---|---|---|---|---|
| | | | | % of |
| | | | Amount per | Tablet* |
| Item | Component | Function | Tablet (mg) | (w/w) |

Famotidine Core Tablet

| | | | | |
|---|---|---|---|---|
| 1 | Famotidine, USP | Active | 26.6 | 2.1 |
| 2 | Microcrystalline | Binder | 44.1 | 3.5 |
| | cellulose, NF | | | |
| | (Avicel PH102) | | | |
| 3 | Lactose, anhydrous | Binder | 12.7 | 1.0 |
| | (SuperTab 21AN), NF | | | |
| 4 | Croscarmellose sodium, | Disintegrant | 4.7 | 0.4 |
| | NF (Ac-Di-Sol SD-711) | | | |
| 5 | Colloidal silicon dioxide, | Glidant | 0.5 | 0.04 |
| | NF (Cab-O-Sil M5P) | | | |
| 6 | Magnesium stearate, NF | Lubricant | 1.4 | 0.1 |

| | | |
|---|---|---|
| Total weight | 90.0 | 7.2 |

Famotidine Core Tablet Coating

| | | | |
|---|---|---|---|
| Opadray white | Coating agent | 5.0** | 0.4 |
| (YS-1-7003) | | | |
| Purified water, USP | Solvent | *** | *** |

| | | |
|---|---|---|
| Total weight | 95.0 | 7.6 |

Ibuprofen Outer Tablet

| | | | | |
|---|---|---|---|---|
| 7 | Ibuprofen DC 85 | Active granules | 941.2 | 75.1 |
| 8 | Microcrystalline | Binder | 111.1 | 8.9 |
| | cellulose, NF | | | |
| | (Avicel PH101) | | | |
| 9 | Povidone, USP | Binder | 55.5 | 4.4 |
| | (Kollidon 30) | | | |
| 10 | Magnesium stearate, NF | Lubricant | 2.8 | 0.2 |

| | | |
|---|---|---|
| Total weight | 1110.6 | 88.6 |

Table-in-Tablet Drug Product

| | | |
|---|---|---|
| Total weight | 1205.6 | 96.2 |

Tablet-in-Tablet Drug Product Coating

| | | | | |
|---|---|---|---|---|
| 6 | Opadry II blue | Coating agent | 48.2**** | 3.8 |
| | (85F99093) | | | |
| 7 | Purified Water, USP*** | Solvent | *** | *** |

| | | |
|---|---|---|
| Total coated tablet-in tablet weight | 1253.8 | 100% |

NF = National Formulary;
USP = United States Pharmacopoeia
*Percent of the total weight.
**Tablets are coated to a weight gain target of 5.5% of total; amount represents solids remaining after drying.
*** Solvent is removed during the processing.
****The finished tablets are coated to a weight gain of 4.0%. The amount represents the solids left after drying.

Stability of a tablet-in-tablet composition containing a famotidine core, Opadry white barrier layer, ibuprofen shell, and Opadry II blue overcoat was tested at 25° C./60% RH for 0, 3, 6, and 9 months. The tablets contained the same active ingredients as the tablets described in Example 1. The stability results of three batches of tablets based on the total % impurities present at each time point are shown below.

Total Impurities Stability Profile of the Tablet-in-Tablet Formulation
90-ct at 25 ± 2° C./60 ± 5% RH

Stability Interval
(Months)

| | | | |
|---|---|---|---|
| Initial | 3 | 6 | 9 |

| | |
|---|---|
| Lot | Total Impurities (%) |

| | | | | |
|---|---|---|---|---|
| 001B | 0.2 | 0.1 | 0.2 | 0.2 |
| 004B | 0.1 | 0.1 | 0.2 | 0.3 |
| 005B | 0.1 | 0.1 | 0.2 | 0.2 |

In addition, the tablet-in-tablet plus barrier layer formulation was compared to a formulation without the barrier layer, as well as a formulation containing coated famotidine granules compressed within ibuprofen. Both sulfamide and total impurities were measured after 1 week at 50° C. and 1 month at 40° C. Under the test conditions, the tablet-in-tablet with barrier layer showed lowered levels of sulfamide formation as shown below.

Stability Evaluation of Tablet-in-Tablet Prototypes

| | | Famotidine | | Ibuprofen | | |
|---|---|---|---|---|---|---|
| | | As- | Sulfamide | | Total | Total |
| Lot | Condition/ | say | Impurity | Assay | Impurities | Impurities |
| No | Time Point | (%) | (%) | (%) | (%) | (%) |

Tablet-in-tablet (DC85 + DC Famotidine w/barrier layer)

| | | | | | | |
|---|---|---|---|---|---|---|
| 014 | Initial | 101.0 | 0.0 | 98.6 | 0.2 | 0.2 |
| | 1 wk 50° C. | 98.4 | 0.19 | 98.8 | 0.1 | 0.3 |
| | 1 mo 40° C. | 96.7 | 0.0 | 100.8 | 0.0 | 0.0 |

Tablet-in-tablet (DC85 + DC Famotidine w/o barrier layer)

| | | | | | | |
|---|---|---|---|---|---|---|
| 015 | Initial | 98.1 | 0.0 | 99.1 | 0.1 | 0.1 |
| | 1 wk 50° C. | 95.4 | 0.74 | 98.3 | 0.1 | 0.9 |
| | 1 mo 40° C. | 94.6 | 0.56 | 99.5 | 0.1 | 0.7 |

Bilayer (DC85 + coated famotidine granules)

| | | | | | | |
|---|---|---|---|---|---|---|
| 016 | Initial | 105.3 | 0.0 | 99.2 | 0.0 | 0.0 |
| | 1 wk 50° C. | 106.7 | 1.28 | 98.9 | 0.1 | 1.4 |
| | 1 mo 40° C. | 103.2 | 0.91 | 100.5 | 2.0 | 3.0 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A pharmaceutical composition, comprising:
a first compartment comprising from 24 mg to 28 mg famotidine as an active pharmaceutical ingredient; and
a second compartment comprising from 750 mg to 850 mg ibuprofen as an active pharmaceutical ingredient,
wherein the pharmaceutical composition is formulated for immediate release, and wherein none of the composition, the first compartment, the second compartment, the famotidine active pharmaceutical ingredient, or the ibuprofen active pharmaceutical ingredient is enterically coated or formulated for sustained or delayed release,
wherein the pharmaceutical composition is formulated so that release of the ibuprofen active pharmaceutical ingredient and the famotidine active pharmaceutical ingredient begins to occur at about the same time, and
wherein no more than about 1% of a sulfamide is present when the composition is stored at 40° C. and 75% relative humidity for a period of one month or at least 90% of the amount of ibuprofen initially present and at least 90% of the amount of famotidine initially present remains after the composition is stored at 40° C. and 75% relative humidity for a period of one month.

2. A pharmaceutical composition of claim 1, wherein the first compartment and the second compartment are separated by a barrier layer.

3. A pharmaceutical composition of claim 1, wherein the composition is a tablet-in-tablet formulation wherein the second compartment completely surrounds the first compartment.

4. A pharmaceutical composition comprising:
from 750 mg to 850 mg ibuprofen as an active pharmaceutical ingredient and
from 24 mg to 28 mg famotidine as an active pharmaceutical ingredient,
wherein the pharmaceutical composition is in the form of a bilayer tablet,
wherein the ibuprofen active pharmaceutical ingredient is present in a first layer and the famotidine active pharmaceutical ingredient is present in a second layer,
wherein the pharmaceutical composition is formulated for immediate release, and wherein none of the composition, the first layer, the second layer, the famotidine active pharmaceutical ingredient, or the ibuprofen active pharmaceutical ingredient is enterically coated or formulated for sustained or delayed release,
wherein the pharmaceutical composition is formulated so that release of the ibuprofen active pharmaceutical ingredient and the famotidine active pharmaceutical ingredient begins to occur at about the same time, and
wherein no more than about 1% of a sulfamide is present when the composition is stored at 40° C. and 75% relative humidity for a period of one month or at least 90% of the amount of ibuprofen initially present and at least 90% of the amount of famotidine initially present remains after the composition is stored at 40° C. and 75% relative humidity for a period of one month.

5. A pharmaceutical composition of claim 4, wherein the first layer and the second layer are separated by a barrier layer.

6. A method for reducing the risk of developing ibuprofen-induced ulcers in a human patient requiring ibuprofen for an ibuprofen-responsive condition, said method comprising:
administering to the human patient a first dose of famotidine,
administering to the human patient a second dose of famotidine, and
administering to the human patient a third dose of famotidine, and
wherein for each administration, the famotidine is administered as a pharmaceutical composition of claim 1.

7. The method of claim 6, wherein said human patient is at elevated risk of developing ibuprofen-induced ulcers.

8. The method of claim 7, wherein said human patient is also being administered low dose aspirin.

9. A method for reducing the risk of developing ibuprofen-induced ulcers in a human patient requiring ibuprofen for an ibuprofen-responsive condition, said method comprising:
administering to the human patient a first dose of famotidine,
administering to the human patient a second dose of famotidine, and
administering to the human patient a third dose of famotidine, and
wherein for each administration, the famotidine is administered as a pharmaceutical composition of claim 2.

10. The method of claim 9, wherein said human patient is at elevated risk of developing ibuprofen-induced ulcers.

11. The method of claim 10, wherein said human patient is also being administered low dose aspirin.

12. A method for reducing the risk of developing ibuprofen-induced ulcers in a human patient requiring ibuprofen for an ibuprofen-responsive condition, said method comprising:
administering to the human patient a first dose of famotidine,
administering to the human patient a second dose of famotidine, and
administering to the human patient a third dose of famotidine, and
wherein for each administration, the famotidine is administered as a pharmaceutical composition of claim 4.

13. The method of claim 12, wherein said human patient is at elevated risk of developing ibuprofen-induced ulcers.

14. The method of claim 13, wherein said human patient is also being administered low dose aspirin.

15. A method for reducing the risk of developing ibuprofen-induced ulcers in a human patient requiring ibuprofen for an ibuprofen-responsive condition, said method comprising:
administering to the human patient a first dose of famotidine,
administering to the human patient a second dose of famotidine, and
administering to the human patient a third dose of famotidine, and
wherein for each administration, the famotidine is administered as a pharmaceutical composition of claim 5.

16. The method of claim 15, wherein said human patient is at elevated risk of developing ibuprofen-induced ulcers.

17. The method of claim 16, wherein said human patient is also being administered low dose aspirin.

18. A pharmaceutical composition in the form of a capsule, wherein the capsule is filled with:
barrier-coated granules comprising from 24 mg to 28 mg famotidine as an active pharmaceutical ingredient and
from 750 mg to 850 mg ibuprofen as an active pharmaceutical ingredient.
